(19) United States
(12) Patent Application Publication
(10) Pub. No.: US 2004/0204499 A1
(43) Pub. Date: Oct. 14, 2004

(54) Preparation for breast duct fluid collection

(76) Inventors: David Hung, Belmont, CA (US); Tina J. Patel, San Carlos, CA (US)

Correspondence Address: CYTYC CORPORATION, 85 SWANSON ROAD, BOXBOROUGH, MA, 01719, US

(21) Appl. No.: 10/775,366

(22) Filed: Feb. 10, 2004

Related U.S. Application Data
(63) Continuation of application No. 09/876,144, filed on Jun. 8, 2001, now abandoned.
(60) Provisional application No. 60/210,438, filed on Jun. 8, 2000.
(60) Provisional application No. 60/236,506, filed on Sep. 29, 2000.
(60) Provisional application No. 60/252,090, filed on Nov. 21, 2000.

(51) Int. Cl. A61K 31/137; A61M 31/00

(52) U.S. Cl. 514/651; 604/500

(57) ABSTRACT

Methods, compositions and systems for preparing a breast for breast duct fluid collection are described.

### CROSS-REFERENCES TO RELATED APPLICATIONS

This application claims the benefit under 37 CFR 1.78 of provisional application 60/210,438 filed on Jun. 8, 2000, provisional application 60/236,506 filed Sep. 29, 2000, and provisional application 60/252,090 filed Nov. 21, 2000. The full disclosures of each of the prior applications are incorporated herein by reference.

### BACKGROUND

Nipple aspiration has been performed on non-lactating women and the material collected from the breast ducts has been analyzed. It is generally thought that the breast contains 6-9 breast ducts and that breast cancer begins in the lining of the breast ducts. Fluid collection from breast ducts has been facilitated by nipple aspiration and to a limited extent by accessing a specific ductal network through a ductal orifice. These procedures have previously been performed with little or no preparation of the nipple surface to help facilitate ductal access or fluid retrieval or both. Thus, it would be advantageous to adequately prepare a breast, nipple surface and/or a breast duct or breast ductal orifice for breast duct fluid collection procedures.

### SUMMARY OF THE INVENTION

The invention provides a composition to contact a breast nipple and to prepare a breast for ductal fluid collection comprising, in bioactive amounts, two or more of the following: an anesthetic agent, a detergent, an exfoliating agent, an antiseptic agent, a dekeratinyzing agent, an orifice-dilating agent, a vaso-dilator, a muscle-relaxing agent, muscle-constricting agent, a lactation-stimulating agent, a secretion-stimulating agent, a sphincter-relaxer, an anti-ischemic agent, a beta-blocker, a calcium channel blocker, a dye or stain to mark the nipple surface excluding ductal orifices, a dye or stain to mark a perimeter of a ductal orifice, and a dye or stain to mark a ductal orifice.

The invention also provides a method of preparing a breast for ductal fluid collection comprising: contacting the nipple surface with a composition comprising an anesthetic and one or more of a detergent, an exfoliating agent, an antiseptic agent, a dekeratinyzing agent, an orifice-dilating agent, a vaso-dilator, a muscle-relaxing agent, a muscle-constricting agent, a lactation-stimulating agent, a secretion-stimulating agent, a sphincter-relaxer, an anti-ischemic agent, a beta-blocker, a calcium channel blocker, a dye or stain to mark the nipple surface excluding ductal orifices, a dye or stain to mark a perimeter of a ductal orifice, and a dye or stain to mark a ductal orifice for sufficient time to allow the composition to take effect on the nipple surface.

The invention provides also a method of preparing a breast duct for access and ductal fluid collection comprising: contacting a ductal orifice with a tip of a ductal access tool coated with a composition comprising one or more of an anesthetic, a detergent, an exfoliating agent, an antiseptic agent, a dekeratinyzing agent, an orifice-dilating agent, a vaso-dilator, a muscle-relaxing agent, a muscle-constricting agent, a lactation-stimulating agent, a secretion-stimulating agent, a sphincter-relaxer, an anti-ischemic agent, a beta-blocker, a calcium channel blocker, a dye or stain to mark the nipple surface excluding ductal orifices, a dye or stain to mark a perimeter of a ductal orifice, and a dye or stain to mark a ductal orifice.

The invention provides a method of preparing a breast duct for access and ductal fluid collection comprising: contacting a ductal orifice with a tip of a ductal access tool coated with a composition comprising one or more of an anesthetic, a detergent, an exfoliating agent, an antiseptic agent, a dekeratinizing agent, an orifice-dilating agent, a vaso-dilator, a muscle-relaxing agent, a muscle-constricting agent, a lactation-stimulating agent, a secretion-stimulating agent, a sphincter-relaxer, an anti-ischemic agent, a beta-blocker, a calcium channel blocker, a dye or stain to mark the nipple surface excluding ductal orifices, a dye or stain to mark a perimeter of a ductal orifice, and a dye or stain to mark a ductal orifice; and infusing a solution comprising an anesthetic into the duct through a lumen of the ductal access tool.

The invention provides a method of preparing a breast duct for access and ductal fluid collection comprising infusing a solution comprising an anesthetic and one or more of an oncotic agent, an osmotic agent, oxytocin, prolactin, a ductal orifice-dilating agent, a. vaso-dilator, a vaso-constricter, a muscle-relaxant, a muscle-constricter, an anti-ischemic agent, a beta-blocker, a calcium channel blocker, a dye or stain to mark the nipple surface excluding ductal orifices, a dye or stain to mark a perimeter of a ductal orifice, and a dye or stain to mark a ductal orifice.

The invention provides a system comprising: an aliquot of a bioactive composition comprising a mixture of one or more of an anesthetic, a detergent, an exfoliating agent, an antiseptic agent, a dekeratinizing agent, an orifice-dilating agent, a vaso-dilator, a muscle-relaxing agent, a muscle-constricting agent, a lactation-stimulating agent, a secretion-siimulating agent, a sphincter-relaxer, an anti-ischemic agent, a beta-blocker, a calcium channel blocker, a dye or stain to mark the nipple surface excluding ductal orifices, a dye or stain to mark a perimeter of a ductal orifice, and a dye or stain to mark a ductal orifice, m a formulation to contact a nipple surface.

The system include that the formulation comprises a powder, a viscous semi-liquid, a foam, a gel, a liquid, or a gas. The system can further include a pad of a geometry to circumscribe a breast nipple to enclose a bioactive agent in contact with a nipple surface and cover the nipple surface for sufficient time for the bioactive agent to act on the nipple surface.

The invention provides a system comprising a ductal access tool and a composition of a formulation capable of coating a tip of the tool to contact a ductal orifice, said composition comprising one or more of an anesthetic, a detergent, an exfoliating agent, an antiseptic agent, a dekeratinizing agent, an orifice-dilating agent, a vaso-dilator, a muscle-relaxing agent, a muscle-constricting agent, a lactation-stimulating agent, a secretion-stimulating agent, a sphincter-relaxer, an anti-ischemic agent, a beta-blocker, a calcium channel blocker, a dye or stain to mark the nipple surface excluding ductal orifices, a dye or stain to mark a perimeter of a ductal orifice, and a dye or stain to mark a ductal orifice.

The invention provides also a system comprising a ductal access tool preloaded with a solution comprising an anesthetic for infusion into an accessed breast duct and a composition of a formulation capable of coating a tip of the tool comprising one or more of an anesthetic, a detergent, an exfoliating agent, an antiseptic agent, a dekeratinizing agent, an orifice-dilating agent, a vaso-dilator, a muscle-relaxing agent, a muscle-constricting agent, a lactation-stimulating agent, a secretion-stimulating agent, a sphincter-relaxer, an anti-ischemic agent, a beta-blocker, a calcium channel blocker, a dye or stain to mark the nipple surface excluding ductal orifices, a dye or stain to mark a perimeter of a ductal orifice, and a dye or stain to mark a ductal orifice.

The invention provides also a method of preparing a patient for a breast duct fluid collection comprising one or more of applying acupuncture, directing meditation, playing music, applying heat to the breast, warming a room where the patient waits, warming a table or chair where the patient lies or sits, covering the patient with a warm blanket.

### BRIEF DESCRIPTION OF THE DRAWINGS

FIG. 1 is a cross-sectional view of a nipple, lactiferous sinus, ductal network, and human breast. The ductal orifice on the nipple surface can be contacted with a probe-like member having a composition coating its tip that transfers some of the composition to the ductal orifice.

FIG. 2 is a cross-sectional view of a nipple, lactiferous sinus, ductal network and human breast being accessed with a ductal access device. The figure depicts infusion of a liquid into the duct from the lumen of the ductal access device.

FIG. 3 is a cross-sectional view of a nipple, lactiferous sinus, ductal network and human breast being accessed with a ductal access device. The device has infused fluid and has nearly filled the duct from a position distal to the ductal sphincter of the lactiferous sinus.

### DESCRIPTION OF THE SPECIFIC EMBODIMENTS

The following examples are offered by way of illustration, not by way of limitation.

The composition to contact the breast nipple prepares the breast for ductal fluid collection. The composition can have, in bioactive amounts two or more agents. A bioactive amount may be a different amount for different agents. In general a bioactive amount can be that amount that is effective on the nipple surface or at the ductal orifice to achieve at least some of the bioactivity expected from the agent. Thus, a bioactive amount should provide enough of the agent for the agent to act on the nipple surface, at a ductal orifice or in a duct or in the breast contacted to a sufficient degree of activity as defined by the purpose of the agent. For example, an anesthetic can work sufficiently in a sufficient amount to provide some level of anesthesia to the tissue contacted. A detergent can act sufficiently in a sufficient amount to clean a nipple surface of molecular and cellular debris and dirt. An exfoliating agent can act sufficiently in a sufficient amount to exfoliate the nipple surface. An antiseptic agent can act sufficiently in a sufficient amount remove the organisms of septic interactions on the nipple surface and at any ductal orifices. A dekeratinizing agent can act sufficiently in a sufficient amount to dekeratinize a nipple surface, particularly with respect to removing keratin molecules at a ductal orifice. An orifice-dilating agent can act sufficiently in a sufficient amount to dilate one or more ductal orifices. A vaso-dilator can act sufficiently in a sufficient amount to dilate one or more blood vessels on the nipple surface, in a duct, or in the breast. A muscle-relaxing agent can act sufficiently in a sufficient amount to relax the muscles in or near the contacted region, including but not limited to the sphincter muscle, and any smooth muscle in the duct and breast. A muscle-constricting agent can act sufficiently in a sufficient amount to constrict any muscles in or near the contacted region. A lactation-stimulating agent can act sufficiently in a sufficient amount to stimulate lactation or secretion in the breast. A secretion-inducing agent can act sufficiently in a sufficient amount to stimulate secretion of breast duct fluid in the breast. A sphincter-relaxer can act sufficiently in a sufficient amount to relax one or more lactiferous sphincters in a corresponding breast duct. An anti-ischemic agent can act sufficiently in a sufficient amount to reduce ischemia in any contacted tissue or in the breast. A beta-blocker can act sufficiently in a sufficient amount to act on the breast to increase blood flow in and near the contacted area and the breast. A calcium channel blocker can act sufficiently in a sufficient amount to act on the breast to increase the blood flow in and near the contacted area and the breast. A dye or stain to mark the non-ductal orifice regions of a nipple surface can act to highlight non-ductal orifice regions of the nipple surface, excluding those regions from interest and targeting in ductal access. A dye or stain to mark a ductal orifice can act to mark one or more ductal orifices on the nipple surface. A dye or stain to mark regions at the perimeter of ductal orifices, can act to mark regions around ductal orifices, so identifying potential target orifices for ductal access. The dye or stain in any event can contain a ligand that preferentially binds a marker to identify either the non-ductal orifice regions of the nipple surface, the perimeter regions of a ductal orifice, or a ductal orifice. More than one dye or stain can be used, e.g. a blue dye that identifies non-ductal orifice regions and a red dye that identifies ductal orifices.

The anesthetic agent can be any anesthetic agent capable of anesthetizing a region of the nipple surface, a ductal orifice, a breast duct, a breast duct system, or a breast. The anesthetic can act topically, systemically, locally, or any combination of these. The anesthetic can comprise but is not limited to, for example, the following mostly topical anesthetics: lidocaine, prolocalne, prevericaine, or marcaine. The anesthetic can also be a combination of mixture of anesthetic agents.

A detergent can be a detergent for cleaning the nipple surface and or a ductal orifice. The detergent may clean a nipple surface, and/or prepare the nipple surface for more effective access and bioactivity by other agents in the composition. The detergents may be a combination of detergents. The detergent can comprise but is not limited to one or more of the following, a salt, a bicarbonate, an oxide, a peroxide, a soap, a liquid detergent such as hand dishwashing detergent, a powder or liquid detergent such a machine dishwashing detergent. The detergent strength can be anything from mild to medium to strong. The detergent and any of the other bioactive agents can foam, bubble or otherwise change character while engaged in bioactivity on the nipple surface.

An exfoliating agent can be an agent capable of removing epidermal cells from the nipple surface. Exfoliating agents act to remove dead or dying epidermal cells from the nipple surface. Exemplary exfoliating agents can include, but are not limited to, for example: agents comprising salicyclic acid, coal tar, zinc, selenium, oatmeal, baking soda, compositions comprising the agent benzoyl peroxide, ketaconazole, and orcorticosteroids, and in general any agent capable of removing or sloughing the top layer of epidermis from the nipple surface.

An antiseptic agent can be any agent capable of reducing an opportunity for sepsis on the nipple surface, at the ductal orifice, or in the breast duct as a result of the breast fluid collection procedure. The purpose of placing the antiseptic agent in the region can be prophylactic, in order to prevent or forestall an opportunity for sepsis to develop in the region. Accordingly, the septic agent can be a medicinal alcohol, for example ethyl alcohol or isopropyl alcohol, in amounts safe for administration to human skin, e.g. nipple skin. The antiseptic agent may also be a topical antibiotic, for example a Neosporin, or a bacteriomycin. The antiseptic agent can be also a mixture or combination of two or more antiseptic agents.

A dekeratinizing agent can be an agent capable of removing at least some keratin from the nipple surface or from a ductal orifice. Keratin is believed to reside in some ductal orifices or sebaceous glands on the nipple surface. Chemical removal of some or all of the keratin that resides in a ductal orifice, can provide multiple opportunities in a ductal fluid collection procedure, not the least of which is the opportunity for the ductal fluid to escape from the duct to the nipple surface upon nipple aspiration, or the opportunity for the ductal orifice to be apparent either to the naked eye, or a magnified view of the nipple surface and ductal orifice, to provide direction as to which orifice to access to collect ductal fluid. Dekeratinizing agents can include, but are not limited to, for example: agents comprising salicyclic acid, coal tar, zinc, selenium, oatmeal, baking soda, compositions comprising the agent benzoyl peroxide (e.g. including sulfur and benzoyl peroxide mixtures), ketaconazole, and orcorticosteroids, and in general any agent capable of removing keratin from a nipple surface and/or from a ductal orifice comprising a keratin plug.

An orifice-dilating agent can be an agent capable of promoting dilation of a ductal orifice. For example, the agent can be from the red pepper family of agents, commonly referred to as capsicum. The agent can also be a hormone capable of prompt or delayed reaction at the ductal orifice to dilate the orifice. For example, prolactin, or oxytocin can act to promote ductal orifice dilation.

A vasodilator can be any agent capable of encouraging vasodilation, or the opening of blood vessels to increase blood flow to and within the region contacted. Vasodilators can be, for example vasodilators used in a cardiac context, and or any vasodilator capable of working at the nipple surface, a ductal orifice, breast duct, and/or the breast.

A muscle-relaxing agent can be any agent capable of relaxing muscles found in a breast duct, or at or near the breast region. The muscles found at the ductal orifice and in the breast duct can be smooth muscles. For example-the sphincter muscle can be chemically relaxed. Muscle-relaxing agents can comprise, for example, a smooth muscle-relaxing agent, and can comprise, for example, calcium channel blockers such as nifedipine, or antispasmodics for example ditropan (oxybutinin), urospas, or terbutyline.

A muscle-constricting agent can be any agent capable of constricting muscles found in a breast duct or at or near the breast region. The muscles found at the ductal orifice and in the breast duct can be smooth muscles. For example the sphincter muscle can be chemically constricted.

A lactation-stimulating agent can be any agent capable of stimulating lactation in lactating women. The agent as applied to a nipple surface and breast of a non-lactating woman may act to increase the ductal fluid collectable from the breast duct or a plurality or breast ducts. A lactation-stimulating agent can comprise but is not limited to, for example, oxytocin or prolactin.

A secretion-stimulating agent can be any agent capable of stimulating secretion of fluids and materials from a duct. The secretion-stimulating agent can comprise, but is not limited to, for example oxytocin or prolactin.

A sphincter-relaxer can be any agent capable of relaxing a sphincter muscle, particularly a breast duct sphincter muscle. Thus, the sphincter-relaxer can be a muscle relaxer that is effective on sphincter-type muscles.

An anti-ischemic agent can be any agent capable of preventing or reducing ischemia. The anti-ischemic agent can work in a variety of ways to achieve the anti-ischemic effect, and use of the agent is not limited by its mode of action. An anti-ischemic agent can act to increase a blood and oxygen flow to the breast region and/or the ductal system, and/or one or more breast ductal orifices.

A beta-blocker can be any beta-blocker capable of acting effectively on a breast from application on the nipple surface to increase a blood flow and oxygen flow to a breast region and/or the ductal system and/or one or more breast ductal orifices.

A calcium channel blocker can be any calcium channel blocker capable of acting effectively on a breast from an application of the agent on the nipple surface to increase a blood flow and oxygen flow to a breast region and/or the ductal system and/or one or more breast ductal orifices.

A dye or stain to mark non-ductal orifice regions of the nipple surface can be capable of identifying the non-ductal orifice regions on the nipple surface to the exclusion of the ductal orifices. A dye or stain to mark the perimeter regions of ductal orifices can be capable of identifying a ring or region surrounding a ductal orifice, or more than one ductal orifice. A dye or stain to mark a ductal orifice can be a dye or stain capable of marking a ductal orifice to the exclusion of other regions of the nipple surface. For example, a dye or stain to mark a ductal orifice can comprise a keratin ligand having a fluorescent tag—and after binding a keratin plug at a ductal orifice (and washing off unbound ligand), a fluorescent tag is evident at at least one ductal orifice on the nipple surface, but not on other non-keratinized regions of the nipple surface.

Contacting the nipple surface with a composition can be accomplished using a formulation that facilitates the contact. The formulation of the composition can be designed so that the composition can be effectively applied to the nipple surface. For example, the formulation can be a powder, a liquid, a gas, a cream, a foam, a gel, and the like. The particular formulation used can dictate the most effective way to contact the nipple surface with the composition. For example, a cream can be spread on the nipple or squeezed from a tube, a foam may be sprayed on from a foam dispenser, a gaseous formulation may be sprayed on, a solid may be sprinkled on, and so on.

A method of preparing a breast for ductal fluid collection comprises contacting the nipple surface with a composition comprising an anesthetic and one or more of a detergent, exfoliating agent, an antiseptic agent, a dekeratinizing agent, an orifice-dilating agent, a vaso-dilator, a muscle-relaxing agent, a muscle-constricting agent, a lactation-stimulating agent, a secretion-stimulating agent, a sphincter-relaxer, an anti-ischemic agent, a beta-blocker, a calcium channel blocker, a dye or stain to mark the nipple surface excluding ductal orifices, a dye or stain to mark a perimeter of a ductal orifice, and a dye or stain to mark a ductal orifice. The contact can be made as is appropriate for the formulation of the composition. For example, a cream can contact the nipple surface by spreading the cream on the nipple surface, for example either with a latex-gloved hand or a stick spreader, etc. A foam formulation can be sprayed on the nipple surface, as can an aerated formulation. A powder can be sprinkled, and so on.

The nipple surface can also be prepared for ductal access and subsequent ductal fluid collection by contacting a ductal orifice with a tip of a ductal access tool coated with a composition comprising one or more of an anesthetic, a detergent, an exfoliating agent, an antiseptic agent, a dekeratinyzing agent, an orifice-dilating agent, a vaso-dilator, a muscle-relaxing agent, a muscle-constricting agent, a lactation-stimulating agent, a secretion-stimulating agent, a sphincter-relaxer, an anti-ischemic agent, a beta-blocker, a calcium channel blocker, a dye or stain to mark the nipple surface excluding ductal orifices, a dye or stain to mark a perimeter of a ductal orifice, and a dye or stain to mark a ductal orifice. The formulation of the composition for coating the tip and exterior sidewalls of a ductal access tool can be a viscous formulation to facilitate the composition to adhere to the tool long enough for the tool and adhered composition to make specific contact with the ductal orifice and early portions of the ductal lumen. The tip of the ductal access tool can be used to probe the nipple surface in search of a ductal orifice on the surface. As the probing is being done, the tip of the tool deposits small aliquots of the composition on the nipple surface. When the tip is close to or directly on a ductal orifice, the composition is likewise deposited at the place of contact between the coated tool and the nipple surface or ductal orifice. As the tool is used to probe the nipple surface for a ductal orifice, the tool can be re-coated with the composition to provide adequate composition on the tool for effective distribution of the composition on the nipple surface and at any contacted ductal orifice.

In addition to contacting the ductal orifice with a ductal access tip coated with a preparative composition as described, thereafter or nearly concurrent with the contact between the tip of the coated tool and the ductal orifice, a solution carried in the tool can be infused into the duct. The infused solution can comprise an anesthetic. The anesthetic can be absorbed into the duct and reduce pain during the ductal access procedure. The anesthetic can serve to prepare the duct for a procedure in which ductal fluid is collected from the breast duct. The method of preparation and palliation for ductal access can also be used for other ductal access procedures practiced for purposes other than ductal material collection, for example during ductoscopy, or intra-ductal surgical procedures, for example the removal of intraductal lesions or similar procedures.

A ductal access procedure may also be prepared for by infusing a solution comprising an anesthetic, and one or more of an oncotic agent, an osmotic agent, oxytocin, prolactin, a ductal-orifice dilating agent, a vaso-dilator, a vaso-constrictor, a muscle-relaxant, a muscle-constrictor, an anti-ischemic agent, a beta-blocker, a calcium channel blocker, a dye or stain to mark the nipple surface excluding ductal orifices, a dye or stain to mark a perimeter of a ductal orifice, and a dye or stain to mark a ductal orifice. Such agents in addition to an anesthetic agent can prepare the ductal orifice and breast ductal system for access and fluid and/or material collection from the duct, and/or may prepare the ductal system for any other intraductal procedure.

After preparation of a nipple surface and one or more ductal orifices by contact with one or more bioactive agents, the breast duct may be accessed by any device adequate for that task. The device can be of dimensions to access a duct. One such procedure can include that ductal fluid and material is collected from the duct. Ductal fluid and material can be collected by any means possible to collect ductal fluid including but not limited to spontaneous discharge, nipple aspiration, and lavage or washing of a duct or multiple ducts in the breast. Lavage is accomplished by infusing wash fluid into the duct and collecting the fluid mixed with ductal fluid.

Any ductal fluid collected by any means can contain material from a breast duct. The material can include, but is not limited to, epithelial cells and other cellular, non-cellular, and/or molecular species either routinely or unexpectedly present in a human breast duct. Material from the terminal ductal lobular unit can also be collected in a lavage procedure, as well as materials residing deep within the ductal passages that access the portion of the breast duct close to the nipple surface, depending on the depth of penetration of the wash fluid and the extent to which the fluid that is introduced into the ductal system is successfully retrieved after mixing with fluid and material in the ductal system. The ductal system includes the terminal ductal lobular unit and any tributary ductal passages that connect with or feed into the ductal system leading to the main breast duct that is accessed at the nipple surface.

In parallel and coordinate with the composition and methods described herein is provided a system for preparing a nipple surface for collection of ductal material comprising an aliquot of a bioactive composition comprising a mixture of one or more of an anesthetic, a detergent, an exfoliating agent, an antiseptic agent, a dekeratinizing agent, an orifice-dilating agent, a vaso-dilator, a muscle-relaxing agent, a muscle-constricting agent, a lactation-stimulating agent, a secretion-stimulating agent, a sphincter-relaxer, an anti-ischemic agent, a beta-blocke, a calcium channel blocker, a dye or stain to mark the nipple surface excluding ductal orifices, a dye or stain to mark a perimeter of a ductal orifice, and a dye or stain to mark a ductal orifice, in a formulation to contact a nipple surface. The formulation can comprise a powder, a viscous semi-liquid, a foam, a gel, a liquid, or a gas.

A system comprising a ductal access tool and such a composition to coat the tip of the tool is also provided by the invention. The composition in the system can be of a formulation capable of coating a tip of the tool to contact a ductal orifice, said composition comprising one or more of an anesthetic, a detergent, an exfoliating agent, an antiseptic agent, a dekeratinizing agent, an orifice-dilating agent, a vaso-dilator, a muscle-relaxing agent, a muscle-constricting agent, a lactation-stimulating agent, a secretion-stimulating agent, a sphincter-relaxer, an anti-ischemic agent, a beta-blocker, a calcium channel blocker, a dye or stain to mark the nipple surface excluding ductal orifices, a dye or stain to mark a perimeter of a ductal orifice, and a dye or stain to mark a ductal orifice.

A system is also provided for preparing a breast for ductal access and/or collection of fluid and material from a breast duct comprising a ductal access tool preloaded with a solution comprising an anesthetic for infusion into an accessed breast duct and a composition of a formulation capable of coating a tip of the tool comprising one or more of an anesthetic, a detergent, an exfoliating agent, an antiseptic agent, a dekeratinizing agent, an orifice-dilating agent, a vaso-dilator, a muscle-relaxing agent, a muscle-constricting agent, a lactation-stimulating agent, a. secretion-stimulating agent, a sphincter-relaxer, an anti-ischemic agent, a beta-blocker, a calcium channel blocker, a dye or stain to mark the nipple surface excluding ductal orifices, a dye or stain to mark a perimeter of a ductal orifice, and a dye or stain to mark a ductal orifice.

In addition to specific methods directed to contact with the nipple surface, ductal orifice and breast ducts, is provided a method of preparing a patient generally for a breast duct fluid collection. The method can comprise one or more of applying acupuncture, directing meditation, playing music, applying heat to the breast, warming a room where the patient waits, warming a table or chair where the patient lies or sits, covering the patient with a warm blanket. The method seeks to relax the patient and facilitate maximal collection of ductal fluid and ductal material from. the ductal access or ductal fluid or material collection procedure.

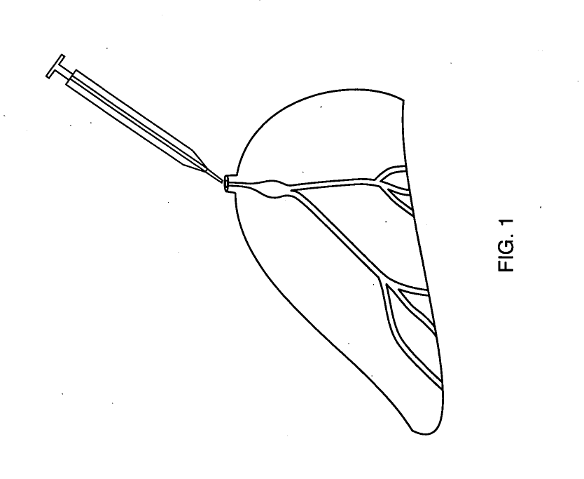

Turning now to the figures, FIG. 1 illustrates a ductal access tool coated with viscous composition comprising a topical anesthetic as it contacts the nipple surface to identify a ductal orifice, and in so contacting the ductal orifice or contacting near the ductal orifice deposits small amounts of the viscous composition that resides on the tool and begins to anesthetize the skin and ductal epithelium just at the orifice. The tool can be redipped into the viscous composition, e.g. where it is taking longer than expected to locate the ductal orifice. The tool may also contain a small amount of liquid anesthetic that can be allowed to drip onto the nipple surface and, where the probe is near an orifice some may enter the orifice and begin anesthetizing the early ductal epithelium close to the orifice.

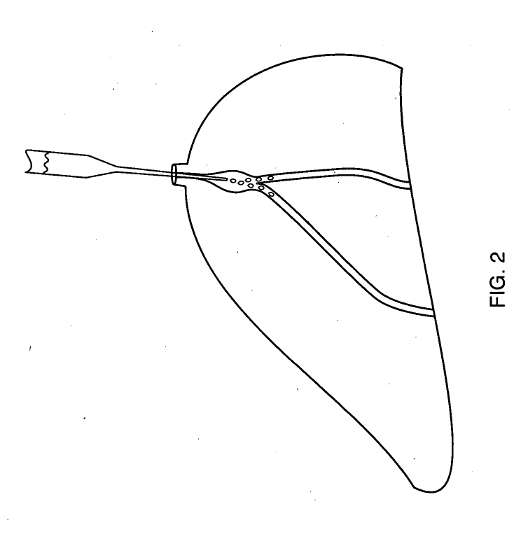

FIG. 2 shows a ductal access tool that has presumably located a ductal orifice, and has penetrated the orifice to access the duct. During the penetration process, presumably the remainder of the viscous composition that remains on the sides of the tool contacts the ductal epithelium at the beginning of the duct as it slides into the duct through the orifice, and also provides anesthetic action in the process. Once accessing the duct, the ductal access tool having a lumen to provide a passage for liquid or fluid or semi-liquid material into the duct can infuse an amount of anesthetic agent into the duct so that the liquid comprising the anesthetic can penetrate into the duct as far as the liquid can travel and thus anesthetize the ductal epithelium that is contacted by the active anesthetic agent.

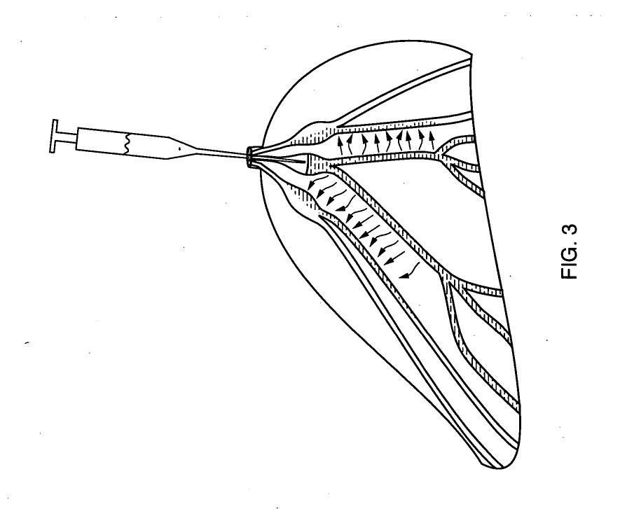

FIG. 3 illustrates that once the duct is filled or substantially filled with liquid comprising anesthetic agent, the agent may contact the ductal epithelium of the penetrated duct and ductal network, and while the invention is not limited to theories of how the invention works, FIG. 3 illustrates the possibility that the anesthetic diffuses through the ductal epithelium and into the breast tissue surrounding the duct and possibly contacting other ductal networks to the extent that their lumens are present in close proximity in the same breast tissue, thus providing anesthetic to a neighboring ducts of the breast including other ductal networks that happen to have lumens within proximity of the accessed duct(s).

### EXAMPLES

### Example 1

The nipple and areola is cleaned with ethanol. Using a magnification loupe, the nipple surface is reviewed by the practitioner. The nipple surface is coated with EMLA™ cream. A ductal access tool as described in U.S. Ser. No. 09/473,510 is primed with about 1 ml (1 cc) of 10 mg/ml liquid lidocaine solution. The distal tip of the tool is dipped in a viscous cream having an active ingredient comprising lidocaine and the tip and sides of the distal region of the tool is coated with a layer of the cream. The tip is then used to probe the nipple surface in regions in which it appears there is a duct. If necessary, the nipple surface can be aspirated to force fluid yield from one or more ducts that can be marked and/or immediately accessed thereafter.

As the probe contacts the nipple surface near an orifice, small droplets of fluid may be released from the access tool lumen to contact the orifice and begin anesthetizing the epithelium cells close to the nipple surface. Once the orifice is penetrated a little bit more liquid lidocaine is released and as the tool penetrates the duct, more lidocaine is dripped into the duct. At a maximum penetration (e.g. from about 5 to about 15 mm) all the lidocaine is released and can then be followed (i.e. chased) by saline. Eventually, the infused saline mixed with ductal fluid and is retrieved, and the collected material is analyzed.

### Example 2

A clinical trial was conducted at several different clinical sites by several different physician practitioners. Sometimes the procedures were conducted by practitioners using topical application of anesthetic, subcutaneous anesthesia injection, sometimes using intraductal anesthesia infusion and sometimes all three. Table 1 shows the results from patients from three different clinical sites, and three different practitioners. In general, conclusions can be made that without subcutaneous injection there was little or no bruising, and that pain of anesthesia administration was greater with subcutaneous injection than with intraductal infusion, or at least that the pain of anesthesia administration and/or the lavage procedure (as subjectively measured by the patients) is no greater with intraductal infusion of anesthetic than it is with subcutaneous injection of anesthetic in a field block. The lavage procedures were conducted essentially as described in the steps 1-15 below.

1. Apply EMLA™ cream (topical anesthetic cream) to the nipple and areola as described in the package insert.

2. Dekeratinize the nipple using gauze and a small amount of Omniprep® paste (dekeratinizing agent) or Keralyt™ gel. Clean the nipple and areola with alcohol and gauze.

3. Before attempting ductal catheterization, identify the ductal orifice under magnification, by using the breast aspirator and/or by gently squeezing on the nipple to yield discharge. Use of an incandescent lamp to warm the nipple may help relax the sphincter, thus making cannulation of the duct easier.

4. For subcutaneous anesthetic administration: using a 10 cc syringe and 18 g needle, withdraw 10 mLs of anesthetic, remove needle and replace with the 30 g needle. Prepare the nipple and areola with a suitable disinfectant and perform nipple field block as needed.

5. Open the catheter package. For intraductal administration of anesthetic, attach anesthetic syringe to inflow (i.e., lower) port and prime the tubing until fluid is expressed through the catheter body and outflow port; leave the syringe attached.

6. Using a new 10 cc syringe and 18 g needle, withdraw 10 mLs of saline (or contrast, if conducting galactography procedure) and set syringe aside.

7. Ensure that approximately 1 cm of the guidewire protrudes from the catheter tip. Under magnification assistance, if necessary, introduce the pre-seated guidewire/catheter unit into the desired ductal orifice to confirm the location and orientation of the duct. Advance the catheter gradually until the catheter is seated; remove the wire completely.

8. Crimp the outflow tubing to prevent return of the anesthetic into the inflow tube. Administer approximately 1 cc of anesthetic through the inflow port (i.e., lower port). Administer more anesthetic as indicated. Disconnect the syringe and set aside for later use. Twist the plunger of a sterile 10 cc syringe to free the seal and attach the syringe to the outflow port.

9. Connect saline filled syringe to the inflow (i.e., lower) port. The catheter is now ready for use.

10. When Performing saline ductal lavage (i.e. fluid infusion and return): Crimp the outflow tubing to prevent early return into the outflow syringe. Using a 10 cc syringe filled with saline, gradually infuse saline in 0.5 cc increments, or until there is slight resistance or some discomfort (typically 2-3 mLs). Release the crimp on the outflow tubing. Massage the breast towards the nipple with both hands for 30 seconds, especially over the infused duct, taking care not to dislodge the catheter. Cloudy fluid and/or small air bubbles may collect in the catheter body, indicating fluid flow. Crimp the inflow tubing and apply suction to the outflow tubing. Release the crimp on the inflow tubing and flush with approximately 1-2 nLs of saline to ensure clearance of infused material into the outflow syringe.

11. Repeat the above step 10. until at least 4 mLs of effluent has collected in the outflow syringe.

12. Remove the catheter from the patient and dispose of appropriately. Collect any residual effluent in a capillary tube to be combined with the lavage sample.

13. Expel effluent into a fresh 15 cc tube containing 8 cc of CytoLyt®. If more than 7 mLs of effluent are recovered, divide the fluid into an appropriate number of tubes (i.e., effluent volume must be less than half of preservative volume). Back thread and twist the cap on tightly. Label the tube with sample identification information. Cut a 1″ strip of Parafilm and stretch it while wrapping it securely around the cap to reinforce the seal.

14. Repeat the above steps for other identified orifices using a new catheter for each orifice.

15. Prepare the specimen for transport in accordance with all applicable regulations for transporting biological materials. Do not freeze the specimen.

Data on lavage procedures were conducted on 57 breasts by 3 practitioners (LE, PS, and RP) and are indicated in Table I below. In Table I, patients are indicated by a number following the practitioner initials. The breast and number of ducts accessed are indicated by an R (right) or L (left), followed by a number indicating the number of ducts accessed. The patients were anesthetized either with topical application of EMLA™, subcutaneous injections of lidocaine (volume indicated 10 mg/ml concentration), or intraductal infusion of lidocaine (10 mg/ml concentration), or all three in some cases. The time for the procedure was indicated in minutes. N/R indicates that the information was not recorded. The patient recorded pain on a scale of 1-100 (1=not painful) for both anesthesia administration and the lavage procedure. These numbers reflect subjective, individual pain recordings, and thus vary in scale and value from patient to patient even with the same practitioner. The “Notes” column that follows indicate notes from the patients' feedback for a period of time from a day to a two weeks after the procedure. Bruising on the breast, pain in the breast, and irritation are the only parameters recorded in Table I in the “Notes” column. Conclusions that can be drawn from the data in Table I include that the highest amount of bruising appears to have occurred with practitioner PS who routinely used the subcutaneous administration of anesthesia in a field block. Lavage pain appeared to be no greater with intraductal administration of anesthetic compared to patients receiving subcutaneous injections of anesthetic, and in some cases pain upon intraductal administration of anesthetic was consistently very low (particularly with practitioner RP). The time of procedure appears to have little or no correlation to anesthesia mode, varying from 10 to 90 minutes, but this recording is not normalized for the number of ducts accessed nor is it rated based on difficulty of access of any one duct (a factor which sometimes affects the overall time of the procedure).

TABLE 1

| | | | | | anes | lav | post |
|---|---|---|---|---|---|---|---|
| | Dr.-patient- | | | | pain | pain | proce- |
| | breast- | subcutan | intraduct | Tim | 1- | 1- | dure |
| # | #ducts | -vol anes | -vol anes | (min) | 100 | 100 | notes |
| 1 | LE-601-R2 | 1 ml | 0 ml | N/R | 8 | 20 | |
| 2 | LE-603-L1 | 2 | 2 | 40 | 93 | 100 | |
| 3 | LE-604-L1 | 5 | 1.5 | 45 | 32 | 0 | |
| 4 | LE-607-R1 | 0 | 2 | 20 | 24 | 1 | |
| 5 | LE-608-L2 | 0 | 2 | 30 | 18 | 31 | bruis- |
| | | | | | | | ing |
| 6 | LE-609-R1 | 0 | 2 | 20 | 60 | 21 | bruis- |
| | | | | | | | ing; |
| | | | | | | | pain |
| 7 | LE-612-R1 | 0 | 2 | 10 | 42 | 1 | |
| 8 | LE-613-R2 | 0 | 2 | 15 | 45 | 16 | |
| 9 | LE-613-L1 | 0 | 2 | 20 | 51 | 22 | |
| 10 | LE-614-R2 | 0 | 2 | 18 | 22 | 2 | |
| 11 | LE-615-R1 | 0 | 3 | 15 | 66 | 14 | |
| 12 | LE-616-R1 | 0 | 2 | 18 | 22 | 47 | |
| 13 | LE-616-L1 | 0 | 3 | 21 | 29 | 82 | |
| 14 | LE-617-L1 | 0 | 2 | 23 | 3 | 72 | |
| 15 | LE-618-R1 | 0 | 2 | 15 | 50 | 34 | |
| 16 | LE-618-L2 | 0 | 2 | 28 | 42 | 27 | |
| 17 | PS-1101-L1 | 5 | 0.5 | 15 | 15 | 1 | bruis- |
| | | | | | | | ing; |
| | | | | | | | pain |
| 18 | PS-1102-R1 | 2 | 0 | 20 | 8 | 9 | bruis- |
| | | | | | | | ing; |
| | | | | | | | pain; |
| | | | | | | | irri- |
| | | | | | | | tation |
| 19 | PS-1103-L1 | 3.5 | 0.5 | 15 | 48 | 69 | bruis- |
| | | | | | | | ing; |
| | | | | | | | pain |
| 20 | PS-1104-L3 | 8 | 0.5 | 50 | 11 | 9 | bruis- |
| | | | | | | | ing |
| 21 | PS-1105-L2 | 10 | 0.5 | 20 | 30 | 8 | bruis- |
| | | | | | | | ing |
| 22 | PS-1106-R2 | 4.5 | 0.5 | 30 | 47 | 27 | |
| 23 | PS-1107-L1 | 4 | 1 | 15 | 8 | 1 | |
| 24 | PS-1108-R2 | 0 | 2 | 30 | 3 | 13 | |
| 25 | PS-1109-R1 | 4 | 1 | 15 | 3 | 1 | |
| 26 | PS-1110-L1 | 2 | 0.5 | 15 | 7 | 1 | bruis- |
| | | | | | | | ing |
| 27 | RP-1802-L1 | 0 | 1 | 25 | 36 | 1 | |
| 28 | RP-1803-L1 | 0 | 1 | 30 | 61 | 61 | |
| 29 | RP-1804-L2 | 0 | 3 | 45 | 1 | 5 | |
| 30 | RP-1805-L1 | 0 | 3 | N/R | 4 | 7 | |
| 31 | RP-1808-R1 | 0 | 1 | 29 | 0 | 0 | |
| 32 | RP-1808-L2 | 0 | 1 | 41 | 1 | 3 | |
| 33 | RP-1809-L2 | 0 | 4 | 50 | 7 | 15 | pain |
| 34 | RP-1810-L1 | 0 | 2 | 25 | 18 | 73 | |
| 35 | RP-1811-R1 | 0 | 3 | 40 | 41 | 0 | pain |
| 36 | RP-1811-L1 | 0 | 3 | 60 | 1 | 2 | |
| 37 | RP-1812-R1 | 0 | 3 | 25 | 9 | 11 | |
| 38 | RP-1812-L1 | 0 | 3 | 35 | 2 | 9 | |
| 39 | RP-1813-R1 | 0 | 3 | 35 | 2 | 24 | |
| 40 | RP-1814-R1 | 0 | 3 | 25 | 44 | 59 | |
| 41 | RP-1814-L1 | 0 | 3 | 20 | 32 | 1 | |
| 42 | RP-1815-L1 | 0 | 3 | 15 | 1 | 1 | |
| 43 | RP-1817-R1 | 0 | 2 | 35 | 2 | 1 | |
| 44 | RP-1818-L1 | 0 | 3 | 15 | 3 | 35 | |
| 45 | RP-1819-R1 | 0 | 3 | 22 | 25 | 7 | |
| 46 | RP-1820-R1 | 0 | 3 | 20 | 26 | 1 | |
| 47 | RP-1820-L1 | 0 | 3 | 20 | 22 | 1 | |
| 48 | RP-1821-R2 | 0 | 2 | 90 | 54 | 32 | |
| 49 | RP-1821-L1 | 0 | 2 | 25 | 22 | 27 | |
| 50 | RP-1822-R1 | 0 | 2 | 30 | 7 | 0 | |
| 51 | RP-1822-L2 | 0 | 3 | 35 | 7 | 1 | |
| 52 | RP-1823-R1 | 0 | 3 | 20 | 85 | 84 | |
| 53 | RP-1823-L1 | 0 | 4 | 25 | 42 | 24 | |
| 54 | RP-1824-R2 | 0 | 5 | 15 | 1 | 1 | |
| 55 | RP-1824-L2 | 0 | 3 | 15 | 1 | 3 | |
| 56 | RP-1825-R1 | 0 | 6 | 15 | 0 | 0 | |
| 57 | RP-1828-L1 | 0 | 3 | 40 | 1 | 1 | |

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. (Canceled)

2. A method of preparing a breast for ductal fluid collection comprising;
contacting the nipple surface of a human breast with a composition comprising an anesthetic and one or more agent selected from the group consisting of a detergent, an exfoliating agent, an antiseptic agent, a dekeratinyzing agent, an orifice-dilating agent, a vaso-dilator, a muscle-relaxing agent, a muscle-constricting agent, a lactation-stimulating agent, a secretion-stimulating agent, a sphincter-relaxer, an anti-ischemic agent, a beta-blocker, a calcium channel blocker, a dye or stain to mark the nipple surface excluding ductal orifices, a dye or stain to mark a perimeter of a ductal orifice, and a dye or stain to mark a ductal orifice for sufficient time to allow the composition to take effect on the nipple surface,
wherein contacting said nipple surface with said composition facilitates ductal fluid collection.

3. A method of preparing a breast ductal for access and fluid collection comprising;
contacting a ductal orifice of a human breast with a tip of a ductal access tool coated with a composition comprising one or more agent selected from the group consisting of an anesthetic, a detergent, an exfoliating agent, an antiseptic agent, a dekeratinyzing agent, an orifice-dilating agent, a vaso-dilator, a muscle-relaxing agent, a muscle-constricting agent, a lactation-stimulating agent, a secretion-stimulating agent, a sphincter-relaxer, an anti-ischemic agent, a beta-blocker, a calcium channel blocker, a dye or stain to mark the nipple surface excluding ductal orifices, a dye or stain to mark a perimeter of a ductal orifice, and a dye or stain to mark a ductal orifice,
wherein contacting said ductal orifice with said composition facilitates ductal access or ductal fluid collection or both.

4. A method of preparing a breast ductal for access and fluid collection comprising;
contacting a ductal orifice of a human breast with a tip of a ductal access tool coated with a composition comprising one or more agent selected from the group consisting of an anesthetic, a detergent, an exfoliating agent, an antiseptic agent, a dekeratinyzing agent, an orifice-dilating agent, a vaso-dilator, a muscle-relaxing agent, a muscle-constricting agent, a lactation-stimulating agent, a secretion-stimulating agent, a sphincter-relaxer, an anti-ischemic agent, a beta-blocker, a calcium channel blocker, a dye or stain to mark the nipple surface excluding ductal orifices, a dye or stain to mark a perimeter of a ductal orifice, and a dye or stain to mark a ductal orifice; and
infusing a solution comprising an anesthetic into the duct through a lumen of the ductal access tool,
wherein contacting said breast duct with said composition facilitates ductal access or ductal fluid collection or both.

5. A method of preparing a breast ductal for access and fluid collection comprising;
infusing a duct of a human breast with a solution comprising an anesthetic and one or more agent selected from the group consisting of an oncotic agent, an osmotic agent, oxytocin, prolactin, a ductal orifice-dilating agent, a vaso-dilator, a vaso-constricter, a muscle-relaxant, a muscle-constricter, an anti-ischemic agent, a beta-blocker, a calcium channel blocker, a dye or stain to mark the nipple surface excluding ductal orifices, a dye or stain to mark a perimeter of a ductal orifice, and a dye or stain to mark a ductal orifice,
wherein infusing said duct with said solution facilitates ductal access or ductal fluid collection or both.

6. (Canceled)
